(19) United States
(12) Patent Application Publication
(10) Pub. No.: US 2004/0034120 A1
(43) Pub. Date: Feb. 19, 2004

(54) Denture adhesive compositions

(76) Inventors: Rajeshwari Patel, Lanham, MD (US); Alan A. Halecky, Glenmoore, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/398,850

(22) PCT Filed: Oct. 10, 2001
(86) PCT No.: PCT/US01/31741

(51) Int. Cl. A61K 6/00

(52) U.S. Cl. 523/120

(57) ABSTRACT

A denture adhesive base composition comprising a multi-carboxylic acid or salt, or mixtures thereof, and ethylene oxide polymer. A denture adhesive composition including this base composition is also provided with other optional adhesive components. Also provided is a method for formulating a novel denture adhesive base composition comprising a multi-carboxylic acid or salt, or mixures thereof, and ethylene oxide polymer.

### FIELD OF THE INVENTION

The present invention is directed to denture adhesives or stabilizers, and particularly to an improved denture adhesive composition.

### BACKGROUND OF THE INVENTION

Traditionally, dentures within the mouth were secured by using adherent powders prepared from natural gum materials such as karaya, acacia or tragacanth gum. These materials have the property of swelling to many times their original volume upon the addition of water to form a gelatinous or mucilaginous mass. Cream forms of the adherent, prepared from finely ground particles of the gums, were also available and used instead of the powder compositions.

Over the years, there have been numerous improvements over the above-described simple denture adhesive formulations. U.S. Pat. No. 2,978,812 discloses a denture fixative composition which includes an ethylene oxide polymer having a molecular weight between 50,000 and 5,000,000 in an amount preferably comprising at least 50% of the active fixative material.

The use of an ethylene oxide polymer, or PEO (“PEO”), has declined over time, being supplanted by compositions which exhibit greater strength. U.S. Pat. No. 3,003,988 describes the use of mixed salts of more than 40 wt. % of a water-insoluble water-sensitized polymeric material consisting essentially of lower alkyl vinyl ether maleic anhydride polymers. U.S. Pat. No. 5,001,170 discloses a substantially anhydrous mixture of about 20-40 wt. % of methyl vinyl ether maleic acid copolymer, 20-40 wt. % of PVP, and 20-40 wt % of ethylene oxide polymer. Recent improvements include the use of a lower alkyl vinyl ether maleic acid, anhydride, or salt polymer or mixtures thereof and one or more metallic salts selected from the group consisting of calcium, magnesium, strontium, sodium, potassium, zirconium, and zinc, or mixtures thereof. U.S. Pat. No. 5,073,604 discloses a denture adhesive composition with mixed partial salts of a lower alkyl vinyl ether maleic acid copolymer, wherein said partial salts contain as the cationic salt function, (a) from about 10% to about 65% zinc or strontium cations; and (b) from about 10% to about 75% calcium cations of the total initial carboxyl groups reacted.

Multi-carboxylic acids and salts thereof are commonly used in the making of disposable diapers and incontinence pads. U.S. Pat. No. 3,707,148 describes the use of carboxylic acids to inhibit microbial growth and ammonia formation in diapers. U.S. Pat. No. 3,964,486 discloses the use of adipic acid and, optionally, an effective amount of sodium adipate in diapers. U.S. Pat. No. 6,010,971 discloses a thermoplastic composition comprising a mixture of a PEO and a multi-carboxylic acid for use in disposable absorbent products and non-woven fibers.

Applicants have surprisingly found that multi-carboxylic acids and salts commonly used in the diaper and absorbent products when combined with PEO in a denture adhesive amount surprisingly improve the cohesive strength of PEO, yielding denture adhesive formulations comparable to formulations based on salts of polymaleic acid methyl vinyl ether in sensory tests as well as cohesive strength, without the need for metal crosslinked polymer systems.

### SUMMARY OF THE INVENTION

A denture adhesive formulation comprising a denture effective amount of PEO and a multi-carboxylic acid or salt, or mixtures thereof. In one embodiment of the invention, the composition comprises from about 1 to 30 wt. % of a multi-carboxylic acid or salt, or mixtures thereof, and from about 5 to 90 wt % PEO. In another embodiment, the composition comprises from about 5 to 15 wt % of a multi-carboxylic acid or salt, or mixtures thereof, and from about 25 to 50 wt % PEO. In one embodiment of a denture liner formulation, the composition comprises from about 5 to 15 wt % of a multi-carboxylic acid or salt or mixtures thereof and from about 30 to 90 wt % PEO.

The invention also relates to a method for adhering a denture to the oral mucosa resulting from the use of the new composition of the present invention.

### DESCRIPTION OF THE DRAWINGS

FIG. 1 is a graph of adhesive force in lbs. vs. the hydration time in minutes, comparing the adhesive composition of the invention with other formulations.

### DETALIED DESCRIPTION OF THE INVENTION

Applicants have unexpectedly discovered a novel denture adhesive composition comprising ethylene oxide polymer and a multi-carboxylic acid or salt, or mixtures thereof, without the need for methyl vinyl ether maleic acid, anhydride, or salt polymers, or the need for an additional processing step for cross-linking these polymer systems with metal ions.

The first component of the adhesive formulation is PEO. PEOs are water soluble, non-ionic, polyether homopolymers having molecular weights from about 100,000 to about 5,000,000. The homopolymers are white powders which when hydrated develop into a gelatinous mass having adhesive characteristics. The polyether polymers have the chemical structure —(CH₂CH₂₀)ₙ— wherein n represents the degree of polymerization of the polymer and may have a value from about 2,000 to about 100,000. PEOs of the type employed in the present invention are more fully described in “Polyox” 1978, published by Union Carbide Corporation, New York, N.Y., as Technical Bulletin F-44029B. PEOs useful in the invention include linear, water-soluble PEOs, as well as cross-linked PEOs. PEOs are commercially available from Union Carbide Corporation under the tradename POLYOX.

A multi-carboxylic acid is any acid that comprises two or more carboxylic acid groups, and preferably oligomers and polymers containing three or more carboxylic groups. Suitable for use in the present invention are dicarboxylic acids, which comprise two carboxylic acid groups. Suitable multi-carboxylic acids include, but are not limited to, glutaric, adipic acid, sebacic acid, malonic acid, citric acid, succinic acid, pimelic acid, suberic acid, azelaic acid, and mixtures thereof.

The multi-carboxylic acids suitable for use in the present invention are generally water-soluble but generally exhibit a solubility that is relatively slower than that exhibited by PEO. As such, the adding of the multi-carboxylic acid to a PEO generally reduces or slows down the water-solubility of the overall mixture.

In one embodiment, the multi-carboxylic acid used is adipic acid, which is widely available in commercial quantities. Adipic acid has acid groups that are six carbons apart (inclusive of the carbonyl carbon). The acid is prepared by oxidizing cyclohexanol with concentrated nitric acid. In another embodiment, sodium adipate is used.

The above two components, PEO and multi-carboxylic acid or salt are used in safe and adhesively effective amounts, which herein means an amount sufficient to provide adherence to the oral cavity. In general, the composition comprises from about 5 to 90 wt % PEO and about 1 to 30 wt. % of a multi-carboxylic acid or salt, or mixtures thereof. In one embodiment, the denture adhesive composition comprises from about 1 to 15 wt. % of a multi-carboxylic acid or salt, or mixtures thereof, and from about 25 to 50 wt. % PEO. In a second embodiment, the denture adhesive composition comprises from about 5 to 15 wt. % of a multi-carboxylic acid or salt, or mixtures thereof, and from about 35 to 50 wt % PEO. In one embodiment of a denture liner formulation, the composition comprises from about 1 to 15 wt. % of a multi-carboxylic acid, or salt, or mixtures thereof, and 30 to 90 wt. % PEO.

In addition to the two ingredients as indicated above to prepare the adhesive base, the composition may optionally contain other components to aid in enhancing the adhesive nature of the base components, including those commonly known and used in the dental adhesive art.

Methyl vinyl ether maleic acid, anhydride, or salts of the copolymers within, and equivalents thereof, may be included although it is not necessary to do so.

Suitable optional adhesive components include the traditional water-soluble hydrophilic colloids or polymers having the property of swelling upon exposure to moisture to form a mucilaginous mass. These materials include copolymers of maleic acid or maleic anhydride or salts thereof, natural gums, synthetic polymeric gums, synthetic polymers, mucoadhesive polymers, hydrophilic polymers, saccharide derivatives, cellulose derivatives, and mixtures thereof. Examples of such materials include karaya gum, guar gum, gelatin, algin, sodium alginate, tragacanth, chitosan, acrylamide polymers, polyacrylic acid derivatives sold under the trademark Carbopol®, polyvinyl alcohol, polyamines, polyquarternary compounds, polybutenes, silicones, cationic polyacrylamide polymers.

In one embodiment, the optional adhesive components are cellulose derivatives, or salts thereof, such as methylcellulose, sodium carboxymethyl cellulose, hydroxyethyl cellulose, hydropropyl cellulose, hydroxypropylmethyl cellulose, carboxymethyl cellulose. In another embodiment, the optional components are carboxy-methylcellulose, karaya gum, sodium alginate, chitosan, polyvinyl alcohol, and mixtures thereof. In general, the other adhesive components may be present at a level of from about 0 to about 70 wt. %. In one embodiment, they are from about 5 to about 50 wt. %. In another embodiment, they are from about 2 to about 20 wt. %.

In addition to the foregoing materials, the denture adhesive composition may be formulated with additional components well known in the denture adhesive art including waxes, oils, preservatives, flavoring agents, colorants, sweetening agents, viscosity modifiers, and so forth.

The waxes useful in the invention include without limitation, both natural and synthetic waxes and include without limitation animal waxes such as beeswax, lanolin and shellac wax, vegetable waxes such as carnauba, candelilla and bayberry wax, mineral wax such as petroleum waxes including paraffin, and microcrystalline.

The oils useful in the invention include without limitation, mineral oil, vegetable oil such as corn, soybean, cottonseed, castor, palm and coconut oils and animal oil such as fish oil, and oleic acid.

Flavoring agents well known to the denture adhesive art may be added to the compositions of the present invention. These flavoring agents include without limitation, synthetic flavor oils and/or oils derived from plants, leaves, flowers, fruits and so forth, and combinations thereof. Representative flavor oils include, spearmint oil, cinnamon oil, oil of wintergreen (methylsalicylate) and peppermint oils. Also useful are artificial, natural or synthetic fruit flavors such as citrus oil including lemon, orange, grape, lime, and grapefruit, and fruit essences including apple, strawberry, cherry, pineapple, and so forth. The flavoring agent may be a liquid, spray dried, encapsulated, or absorbed on a carrier, and mixtures thereof. A preferred flavoring agent is peppermint oil. The amount of flavoring agent utilized varies depending on such factors as flavor type, adhesive formulation and strength desired. In general, amounts of about 0.01 to about 5.0 wt. % of the total denture adhesive composition are suitable. In one embodiment of the invention, an amount of about 0.05 to 0.15 wt. % is used.

Preservatives which may be used in the denture adhesive formulations of the invention include those known antimicrobial agents conventionally employed in the art, such as benzoic acid and sodium benzoate; the parabens; sorbic acid and sorbates; propionic acid and propionates; acetic acid and acetates; nitrates and nitrites; sulfur dioxide and sulfites; antibiotics; diethyl pyrocarbonate; epoxides; hydrogen peroxide; and phosphates. The parabens include the methyl, ethyl, propyl, and butyl esters of parahydroxybenzoic acid. Methyl paraben and propyl paraben are the preferred preservatives of the invention, utilized in amounts of about 0.03 to about 0.06 wt. % of the total denture adhesive composition.

The denture adhesive compositions may also include the use of sweeteners well known in the art. The sweetening agent may be selected from a wide range of materials including water-soluble agents, water-soluble artificial sweeteners, and dipeptide based sweeteners, including mixtures thereof. Representative sweeteners include without limitation, (a) water-soluble sweetening agents such as monosaccharides, disaccharides and polysaccharides such as xylose, ribose, glucose, mannose, galactose, fructose, dextrose, sucrose, sugar, maltose, partially hydrolyzed starch, or corn syrup solids and sugar alcohols such as sorbitol, xylitol, mannitol, maltitol, hydrogenated starch hydrolysate, and mixtures thereof; (b) water-soluble artificial sweeteners such as the soluble saccharin salts, i.e., sodium or calcium saccharin salts, cyclamate salts, acesulfam-K, sucralose, and the like, and the free acid form of saccharin; and (c) dipeptide based sweeteners such as L-aspartyl-L-phenylalanine methyl ester, and the like. In general, the amount of sweetener may be about 0.001 to about 5 wt. % of the total denture adhesive composition.

The colorants useful in the present invention include the pigments such as titanium dioxide, and may also include dyes suitable for food, drug and cosmetic applications. These colorants are known as FD&C dyes. Illustrative examples include without limitation, indigo dye, known as FD&C Blue No. 2, which is the disodium salt of 5,5′-indigotindi-sulfonic acid; FD&C Green No. 1, comprising a triphenylmethylene dye and is the monosodium salt of the 4[4-N-ethyl-p-sulfobenzylamino) diphenylmethylene]-[1-(N-ethyl-N-P-sulfobenzyl)-2,5-cyclohexadienimine]. A preferred colorant is FD&C Red No. 3.

The viscosity modifiers useful herein include without limitation, quaternary ammonium compounds and similar agents, starches, gums, casein, gelatin and semi-synthetic cellulose.

The composition of the present invention may also be used as a denture adhesive and/or bioadhesive and comprise one or more therapeutic actives suitable for mucosal or topical administration. The phrase “suitable for mucosal or topical administration,” as used herein, describes agents which are pharmacologically active when absorbed through internal mucosal surfaces of the body such as the oral cavity, or applied to the surfaces of the skin. Therapeutic actives may be present at a level from about 0 to about 40 wt. % of the composition.

Therapeutic actives that are useful in the instant compositions include antimicrobial agents such as iodine, sulfonamides, bisbiguanides, or phenolics; antibiotics such as tetracycline, neomycin, kanamycin, metronidazole, or clindamycin; anti-inflammatory agents such as aspirin, acetaminophen, naproxen and its salts, ibuprofen, ketorolac, flurbiprofen, indomethacin, eugenol, or hydrocortisone; dentinal desensitizing agents such as potassium nitrate, potassium chloride, strontium chloride or sodium fluoride; anesthetic agents such as lidocaine or benzocaine; antifungals; aromatics such as camphor, eucalyptus oil, and aldehyde derivatives such as benzaldehyde; insulin; steriods; and anti-neoplastics. It is recognized that in certain forms of therapy, combinations of these agents in the same delivery system may be useful in order to obtain an optimal effect. Thus, for example, an antimicrobial and an anti-inflammatory agent may be combined in a single delivery system to provide combined effectiveness.

The denture adhesive compositions may be in the form of a powder, a paste, a cream, or a liner. Means for preparing such formulations is well known in the denture adhesive art, employing conventional types of mixing equipment for blending, heating, and cooling solids and liquids. In one embodiment, mixing is performed at temperatures suitable to melt the components, e.g., petrolatum, to be blended.

In the powder form, the components are admixed with flavoring agents and colorants, together with other ingredients such as non-toxic anti-caking agents (silica, magnesium stearate, talcum powder, and the like). The mixture of ingredients is thoroughly agitated or stirred to yield a generally homogenous intermixing of all components.

In the cream or paste formulation, the components are admixed with petrolatum along with the previously described waxes, oils, preservatives, flavoring agents, colorants, sweetening agents, viscosity modifiers, and so forth.

In the liner or layer form, the component are uniformly mixed and then coated onto a non-adhesive self supporting coating layer by any conventional coating techniques, such as by spraying (if the material is liquid or slurry or dissolved or suspended in a liquid such as water) or by sifting (if the denture adhesive is in powder form). In another embodiment, the components are admixed with the previously described waxes, oils, preservatives, flavoring agents, colorants, sweetening agents, viscosity modifiers, and so forth. The liner is then formed by any variety of techniques known in the polymer film-forming art, including casting, calendaring, coating, and extrusion. In one embodiment to form liners, the components are first mechanically softened by a ring roller; smoothed on a hydraulic press, and die-cut as desired into denture liner shapes or other desired shapes.

To further illustrate the invention, Examples are set forth below. In these, as throughout the specification and claims, all parts and percentages are by weight and all temperatures in degrees centigrade, unless otherwise indicated.

### EXAMPLES

### Example 1

### Cream Formulation

In this example, PEO and sodium adipate were mixed together and added to the melted petrolatum and mineral oil in a KitchenAid mixer with stirring. Flavors, dyes and other optional ingredients were added and intimately mixed in for about 30 minutes. The resultant denture adhesive composition was a viscous material which turned into a smooth soft mass upon wetting and mixing with water or saliva

| | |
|---|---|
| Sodium Adipate | 10.00 |
| PEO Polyox 301 100 mesh | 42.00 |
| Mineral Oil | 25.75 |
| Petrolatum | 19.70 |
| Optamint N & A peppermint flavor | 0.50 |
| D & C Red #7 | 0.02 |
| D & C Red #30 | 0.03 |
| Total | 100.00 |

### Example 2

### Denture Liner Formulation

For this formulation, PEO powder was first admixed with sodium adipate in a KitchenAid™ mixer and then added to a mixture of CMC and HPMC. The blend was plasticized with an appropriate amount of water and glycerin and extruded forming a film. A typical denture liner formulation is as follows:

| | |
|---|---|
| Sodium Adipate | 5.00 |
| PEG Polyox 301 100 mesh | 45.00 |
| CMC 7H3SXF | 12.50 |
| Glycerin | 5.00 |
| HIPMC (Hydroxypropylmethyl cellulose) | 12.50 |
| Water | 20.00 |
| Total | 100.00 |

### Example 3

### Powder Formulation

For this formulation, PEO powder was mixed with sodium adipate and other ingredients in a blender. A typical denture powder formulation is as follows:

| | |
|---|---|
| Sodium Adipate | 5.00 |
| PEO Polyox 301 100 mesh | 45.00 |
| CMC 7H3SXF | 31.00 |
| HPMC (Hydroxypropylmethyl cellulose) | 19.00 |
| Total | 100.00 |

### Example 4

### Comparative Example with Polyox Only

In this example, PEO was added to melted petrolatum and mineral oil in a KitchenAid™ mixer with stirring. Flavors, dyes and other optional ingredients may be added in the next mixing phase and intimately mixed in for about 30 minutes. The resultant denture adhesive composition was a viscous material which turned into a smooth soft mass upon wetting and mixing with water or saliva.

| | |
|---|---|
| PEO Polyox 301 100 mesh | 45.00 |
| Mineral Oil | 25.00 |
| Petrolatum | 30.00 |
| Total | 100.00 |

### Adhesive Force Evaluation

The cream formulation of the present invention (Example 1) was compared with Comparative Example 4 (with Polyox only and without multi-carboxylic acid/salt) and a commercially available denture adhesive cream formulation Fixodent®, known to contain metal salts of methyl vinyl ether/maleic acid or anhydride copolymers or Gantrez® as disclosed in U.S. Pat. No. 5,073,604.

The samples were evaluated for adhesion characteristics by an adhesive force test called the “Dislodge Force Method.” The test method involves the use of two plates, an upper and a lower plate, in a water bath at about 37° C. The upper plate is lined with felt (representing the Maxilla) and attached to a Chatillon® gauge and moving ram. The lower plate is made out of polymethyl methacrylate (representing the denture) and attached onto a hinge which is attached to a Chatillon® test stand. As the upper plate applies downward pressure, the lower plate swings downward onto the hinge. This action simulates the biting motion of the mouth. The swinging motion of the lower plate simulates the dislodgement of a denture.

In this test, strips of adhesive with about 0.125 grams of adhesive per strip were applied onto the lower plate. The two plates were then brought together under 10 lbs. compression force for 5 minutes. At the end of the 5 minutes, compression was removed and the upper plate was lowered at a rate of 25 mm per minute. A force (representing the first dislodge force reading) was registered by the Chatillon® gauge. During this cycle, the downward swinging motion of the lower plate represented dislodgement. This compression, decompression, and reading cycle was repeated again for 11 more times, giving a total of 12 cycle readings of 5 minutes each, or a total of 60 minutes hydration time. The dislodge force (in lbs.) for the 1^{st}, 2^{nd}, 3^{rd}, . . . , 12^{th} cycle was recorded and then plotted graphically in FIG. 1.

FIG. 1 compares the cream formulation of the present invention (Example 1) with Comparatives Examples 4 ( Polyox only) and the commercially available denture adhesive composition, Fixodent®. The adhesive force evaluation test indicated that the composition of the present invention displayed comparable, if not superior, time hold property to the commercially available denture composition containing metal salts of methyl vinyl ether/maleic acid or anhydride copolymers or Gantrez®.

### Sensory Evaluation

The denture cream formulation in Example 1 was tested against commercially available denture adhesive cream formulation Fixodent® Original, containing metallic crosslinked methy vinyl ether maleic acid copolymer. The sensory tests were carried out to evaluate the organoleptic characteristics of the PEO/multi-carboxylic acid/salt formulation of the present invention against a leading market denture adhesive.

In organoleptic evaluation tests, several expert evaluation panelists were presented with two samples of denture adhesive creams. One was the formulation of the present invention, the other was the commercial formulation. The panelists were asked to complete a questionnaire to evaluate the denture creams based on fifteen different criteria, e.g., mouth feel, hold property, time hold property develops, oozing property, ease to remove, ease to apply, etc. Statistical analyses of the tests indicated that the denture formulation of the present invention was judged to be comparable to the commercial formulations comprising metal salts of methyl vinyl ether/maleic acid or anhydride copolymers of Gantrez®. It was also found that the denture formulation of the present invention did not exhibit any objectionable phase separation or oozing from a dental plate.

The above description fully discloses the invention including preferred embodiments thereof. Modifications and improvements of the embodiments specifically disclosed herein are within the scope of the following claims. Without further elaboration it is believed that one skilled in the art can, given the preceding description, utilize the present invention to its fullest extent. Therefore any examples are to be construed as merely illustrative and not a limitation on the scope of the present invention in any way. The embodiments of the invention in which an exclusive property or privilege is claimed are defined as follows.

What is claimed:

1. A denture adhesive base composition comprising:
(a) from about 5 to 90 wt. % ethylene oxide polymer;
(b) from about 1 to 30 wt. % of a multi-carboxylic acid or salt, or mixtures thereof, and
(c) at least 5 wt. % of additional materials selected from the group consisting of cellulose derivatives, polyacrylic acid derivatives, sodium alginate, waxes, oils, preservative, flavoring agents, colorants, sweetening agents, viscosity modifiers, and mixtures thereof, wherein said composition is capable of adhering to a wet mucous surface.

2. The denture adhesive base composition of claim 1 in a form of a denture liner, wherein said multi-carboxylic acid or salt, or mixture thereof, is about 1 to 15 wt. %, and said ethylene oxide polymer is about 30 to 90 wt. %, based on the total weight of the denture adhesive base composition.

3. The denture adhesive base composition of claim 1, wherein said multi-carboxylic acid or salt, or mixture thereof is about 1 to 15 wt. %, and said ethylene oxide polymer is about 25 to 50 wt. %, based on the total weight of the denture adhesive base composition.

4. The denture adhesive base composition of claim 3, wherein said multi-carboxylic acid is adipic acid in an amount of about 5 to 15 wt. %, based on the total weight of the denture adhesive base composition.

5. The denture adhesive base composition of claim 3, wherein said multi-carboxylic acid is sodium adipate in an amount about 5 to 15 wt. %, based on the total weight of the denture adhesive base composition.

6. The denture adhesive base composition of claim 1 in the form of a cream formulation.

7. The denture adhesive base composition of claim 1 in the form of a denture liner.

8. The denture adhesive base composition of claim 1, further including from about 0 to about 70 wt %, based on the total weight of the denture adhesive composition, of additional materials selected from the group consisting of cellulose derivatives, polyacrylic acid derivatives, sodium alginate, and mixtures thereof.

9. The denture adhesive base composition of claim 8, further including from about 0 to less than 20 wt %, based on the total weight of the denture adhesive composition, of a lower alkyl vinyl ether maleic acid, anhydride, or salt polymer or mixtures.

10. The denture adhesive base composition of claim 8, wherein the cellulose derivatives comprise carboxymethyl cellulose.

11. The denture adhesive base composition of claim 9, further including additional materials selected from the group consisting of waxes, oils, preservatives, flavoring agents, colorants, sweetening agents, viscosity modifiers, and mixtures thereof.

12. The denture adhesive base composition of claim 9, further including non-toxic, powdered, excipient materials.

13. A method for preparing a denture adhesive base composition comprising:
(a) forming a denture adhesive composition from about 5 to 90 wt % ethylene oxide polymer, from about 1 to 30 wt. % of a multi-carboxylic acid or salt, or mixtures thereof, and at least 5 wt % additional materials selected from the group consisting of: cellulose derivatives, polyacrylic acid derivatives, sodium alginate, waxes, oils, preservative, flavoring agents, colorants, sweetening agents, viscosity modifiers and mixtures thereof; and
(b) recovering said denture adhesive composition.

14. The method of claim 13, wherein said multi-carboxylic acid or salt, or mixtures thereof, is about 1 to 15 wt. %, and said ethylene oxide polymer is about 30 to 90 wt. %, based on the total weight of the denture adhesive base composition.

15. The method of claim 13, wherein said denture adhesive base composition further includes up to about 70 wt. %, based on the total weight of the denture adhesive composition, of additional materials selected from the group consisting of cellulose derivatives, polyacrylic acid derivatives, sodium alginate, and mixtures thereof.

16. The method of claim 14, wherein said denture adhesive base composition further includes additional materials selected from the group consisting of waxes, oils, preservatives, flavoring agents, colorants, sweetening agents, viscosity modifiers, and mixtures thereof.

17. A process for the preparation of the denture adhesive base composition of claim 1, wherein the denture adhesive composition is mechanically softened and pressed smooth and cut into denture shapes.

18. Use of a denture adhesive base composition comprising a safe and adhesively effective amount of two components: (a) multi-carboxylic acid or salt, or mixtures thereof; and (b) ethylene oxide polymer as a denture adhesive, wherein said composition is capable of adhering to a wet mucous surface.

19. Use of the denture adhesive base composition of claim 18, further including from about 0 to about 70 wt. %, based on the total weight of the denture adhesive composition, of additional materials selected from the group consisting of cellulose derivatives, polyacrylic acid derivatives, sodium alginate, and mixtures thereof.

20. Use of the denture adhesive base composition of claim 19, further including from about 0 to less than 20 wt. %, based on the total weight of the denture adhesive composition, of a lower alkyl vinyl ether maleic acid, anhydride, or salt polymer, or mixtures thereof.
